(19) United States
(12) Patent Application Publication
(10) Pub. No.: US 2006/0253175 A1
(43) Pub. Date: Nov. 9, 2006

(54) Programmable photo power source

(75) Inventors: Chu-Ping Fan, Hsin-Chu (TW); Hsiang-Cheng Hsieh, Hsin-Chu (TW); Shang-Chih Tsou, Hsin-Chu (TW)

Correspondence Address: TROXELL LAW OFFICE PLLC, SUITE 1404, 5205 LEESBURG PIKE, FALLS CHURCH, VA, 22041, US

(73) Assignee: Lighthouse Technology Co., Ltd.

(21) Appl. No.: 11/304,751

(22) Filed: Dec. 16, 2005

(30) Foreign Application Priority Data
May 6, 2005 (TW) 094207308

(51) Int. Cl. A61N 5/06

(52) U.S. Cl. 607/88; 607/90

(57) ABSTRACT

A programmable photo power source includes a light emitting assembly containing at least two irradiation panels foldable to each other and each comprised of multiple surface-mounted LEDs; a base to secure the assembly; and a control panel to control operation time, hours and current of each surface-mounted LED to produce better irradiation results with preset wavelength programmable through the control panel.

### BACKGROUND OF THE INVENTION

(a) Field of the Invention

The present invention is related to a programmable source to generate photo power, and more particularly, one has light emitting diode (LED) as its source of power.

(b) Description of the Prior Art

Whereas photo power is capable of producing photochemical reaction with photosensitizer in tissue by transferring photo power to photosensitizers resulting in the singlet oxygen production that is toxic to the cell.

So far in medical phototherapy field, many light beams of given wavelength have been taken as the source to excite photosensitizers for the purpose of targeting tumor cells, and even further causing toxicity to the cell where the photosensitizers is located to achieve the purpose of tumor selectivity. Furthermore, results of studies indicate that exposure of the tissues of the wound to the the infrared laser of single wavelength radiation would help to promote healing of the wound and other processes including production of growth factors, synthesis of collagen, formation of extra-cellular matrix, and proliferation of fibroblasts

Early in the medical world, laser beam was the primary source of power for the power therapy. However the purchase price and the maintenance cost were comparatively more expensive and those systems introduced even earlier were too heavy to allow easy movement (The major disadvantage is the power; Because of it's high intensity irradiation, Laser may cause damage to normal cells under misoperation.). Therefore, LED, arc lamp, and incandescent lamp that provide simple construction, cheaper, reliable, higher mobility, and absence of invasive injury have been consecutively developed.

For example, a utility patent published in Taiwan Patent Gazette No. 511514 titled “Acne Curing Irradiator Using Blue and Red LEDs” and another utility patent published in Taiwan Patent Gazette No. 580916 titled “Wound Healing Irradiator using LED” both respectively teach an irradiator or power system having LED as the power source in photo-therapy to correct many flaws of the laser system. In another utility patent yet as published in Taiwan Patent Gazette No. 421993 titled “Plant Growing Box with Ultra High Luminance LED as Man-made Light Source” discloses an art of growing a plant by the application of cold light source from LED, LED is used to improve the energy produced by each cellular mitochondrion (for the energy part) for the cells to grow faster thus to help accelerate the growing of the plant.

FIG. 1 of the accompanying drawings shows a basic construction of a power source of the prior art that has LED as the light source. Wherein, an irradiation panel 21 comprised of multiple LED bulbs 30 is placed over a base 10 and multiple operation keys 40 are disposed on the surface of the base 10 for the user to control the operation of those multiple LED bulbs 30 as required. However, a significant pattern of alternatively brighter and darker areas is created due to the smaller angle of light emission by each LED bulb 30, and when mixed light is desired, the mixed light area becomes even smaller to result in poor mixing as illustrated in FIG. 2. Furthermore, an individual photosensitive substance usually needs a light of specific wavelength to be excited and activated. Therefore, it takes a light source of proper wavelength along with discovery and synthesis of new photosensitive substances. Most of those power sources of the prior are capable of producing only a single wavelength light source, thus their applications are limited.

### SUMMARY OF THE INVENTION

The primary purpose of the present invention is to provide a programmable photo power source includes an assembly of light emitting source comprised of at least two irradiation panels with each including multiple surface mounted LEDs functioning to produce a light source; a base to secure the assembly in position and a control panel is to control operation time, number of hours and current of each LED to provide better irradiation results from those surface-mounted LEDs and control of wavelength of the beam preset through the control panel as desired.

### BRIEF DESCRIPTION OF THE DRAWINGS

FIG. 1 is a perspective view of a photo power source of the prior art.

FIG. 2 is a schematic view showing the status of the irradiation performance from a bulb type of LED.

FIG. 3 is a perspective view of a first preferred embodiment the present invention.

FIG. 4 is a layout of a light emitting source assembly of a preferred embodiment of the present invention.

FIG. 5 is a sectional view of multiple surface-mounted LEDs in another preferred embodiment of the present invention.

FIG. 6 is a schematic view showing the status of the irradiation performance from those surface-mounted LEDs of the present invention.

FIG. 7 is a schematic view showing a heat sink mechanism of the first preferred embodiment of the present invention.

FIG. 8 is a schematic view showing a heat sink mechanism of the second preferred embodiment of the present invention.

FIG. 9 is a schematic view showing a layout of a channel in a heat sink mechanism of the third preferred embodiment of the present invention.

FIG. 10 is a sectional view showing a construction of the channel in the heat sink mechanism of the third preferred embodiment of the present invention.

FIG. 11 is a schematic view showing a layout of a pump in the heat sink mechanism of the third preferred embodiment of the present invention.

FIG. 12 is a schematic view showing the link configuration between a control panel and all related members of the present invention.

FIG. 13 is a schematic view showing the operation time and the size of current for each of those multiple light emitting chips of the present invention.

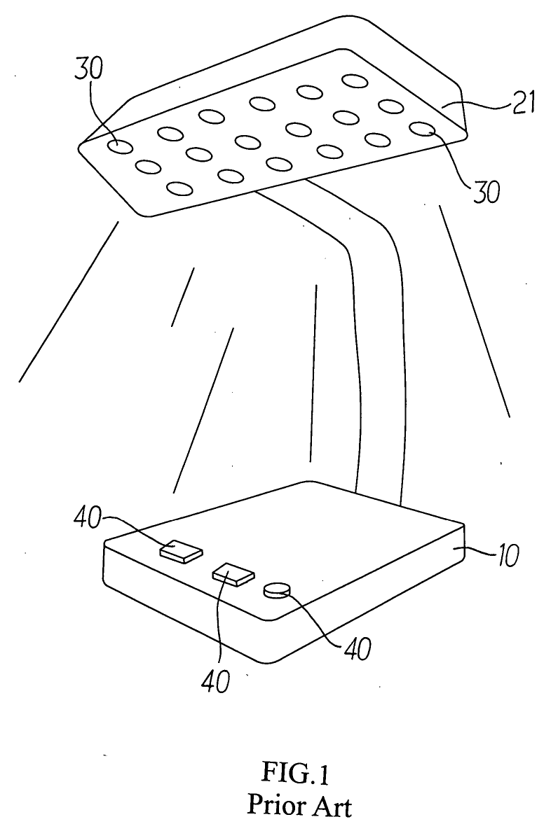

### DETAILED DESCRIPTION OF THE PREFERRED EMBODIMENTS

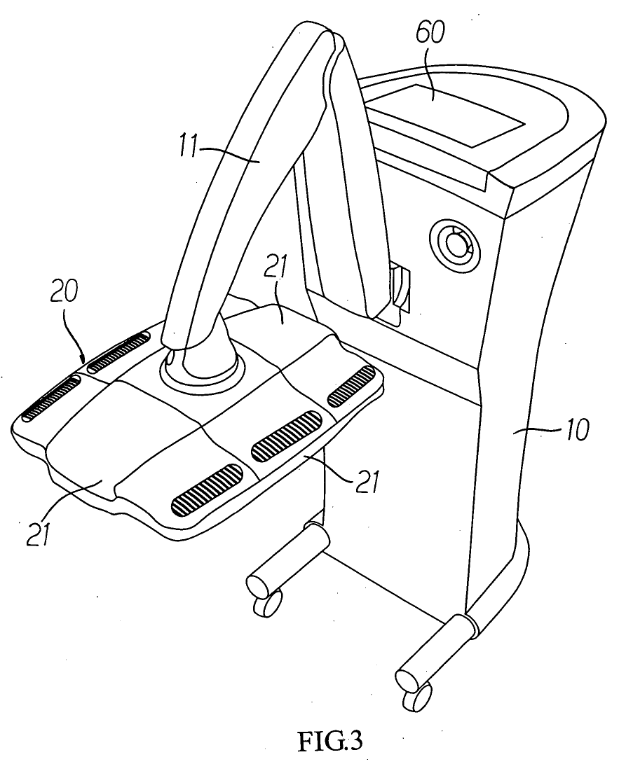

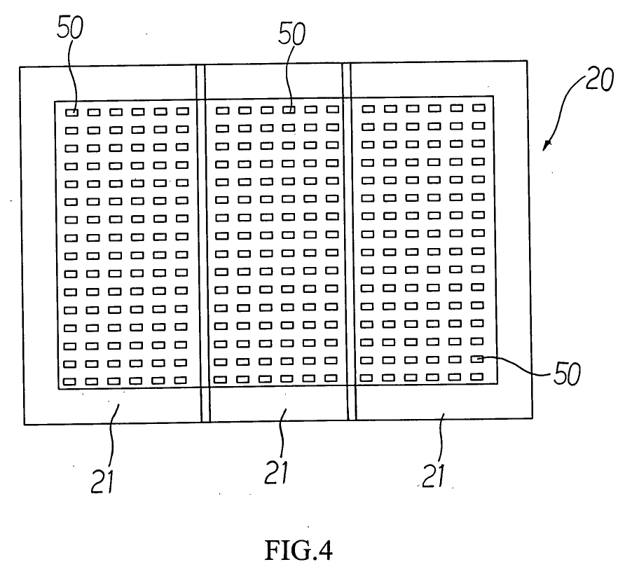

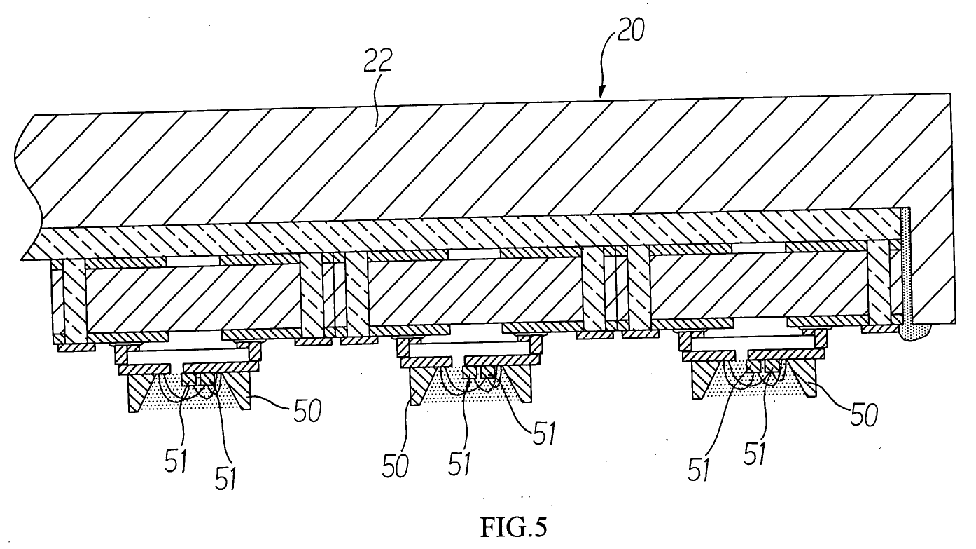

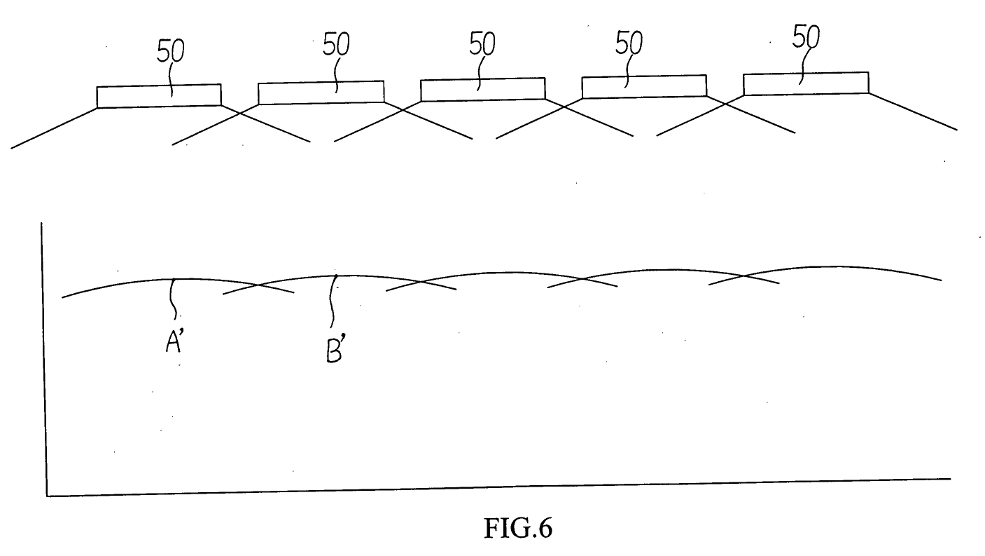

Referring to FIGS. 3 and 4 for a preferred embodiment of the present invention, a programmable photo power source is related to a light emitting source assembly 20 comprised of at least two irradiation panels 21 each including multiple surface-mounted LEDs 50 to generate light source. The assembly 20 is secured on a base 10 and adapted with a control panel 60 to control operation time, hours, current, and wavelength of each of those surface-mounted LEDs 50.

Wherein, one or multiple light emitting chip 51 is built in each LED 50 to permit any single light emitting point within the assembly 20 to provide changes in wavelength of light thus to expand the range of application for the assembly. As illustrated in FIG. 5, a reflective material 22 is provided to each irradiation panel 21 in the assembly 20 to boost the luminance of those LEDs 50. The reflective material 22 together with the inherited performance of greater light emitting angle of those LEDs 50 contributes to the light irradiation effects in a pattern that is less subject to the phenomenon of alternatively brighter and darker areas as illustrated in FIG. 6.

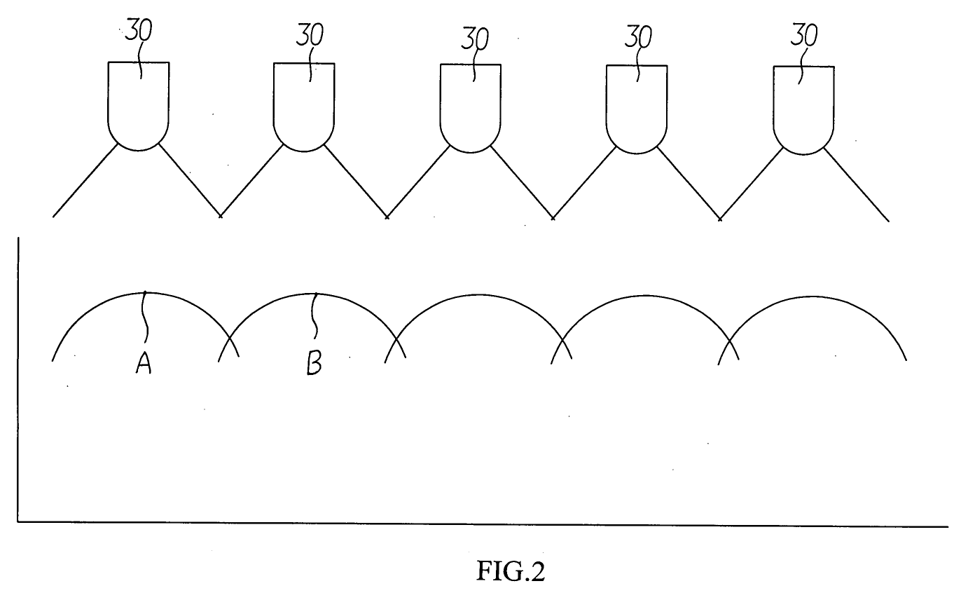

Irradiation results from using a given bulb type of light emitting diodes 30 as illustrated in FIG. 2 are compared with that from its adjacent diode 30 in the mixing of different wavelengths of light. To their respective light mixing areas A and B, the wavelength of the mixed light is not consistent. Wherein, the resultant wavelengths from light mixing areas A and B are respectively close to that of the diode directly above. On the contrary, the mixed wavelength in similar light mixing areas A′ and B′ below their respective surface-mounted LED 50 created by the present invention is more consistent due to that surface-mounted LED 50 provides greater view angle of light emission and contains multiple light emitting chips of different wavelengths as illustrated in FIG. 6.

Now referring to FIG. 3, a support 11 is disposed on the base 10 to help secure the assembly 20 to a certain height where permits the folding between two irradiation panels 21 to adjust for an optimal angle. Each irradiation panel 21 enables the folding within a range of 0° up to 90° as needed by the operator to produce multi-directional irradiation results against the target tissue as illustrated in FIG. 6. Furthermore, the direct mounting of those LEDs 50 to the body of the assembly 20 facilitates the installation of a heat sink 70 at where the irradiation panel 21 is located to avoid affecting the ambient temperature due to longer time of operating the assembly 20.

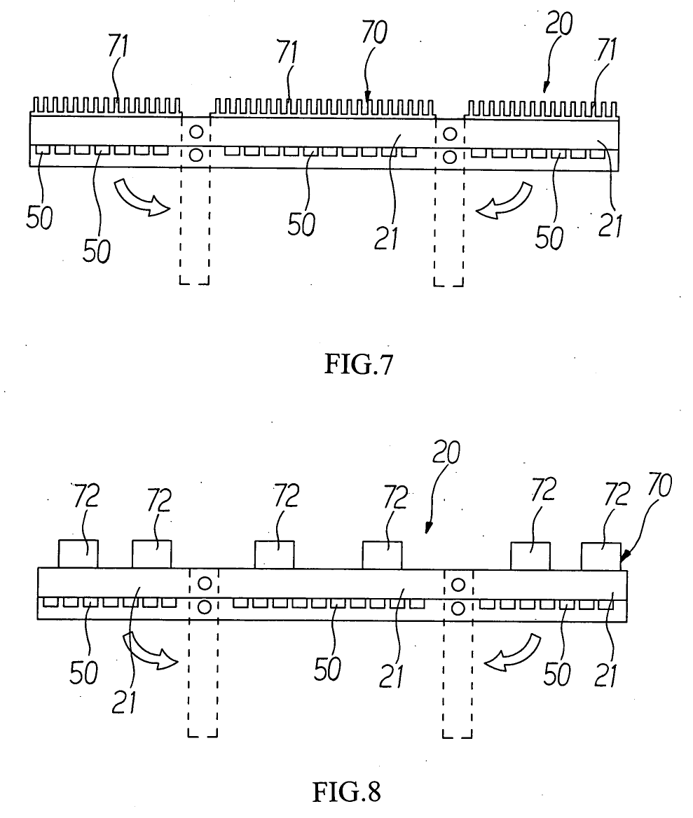

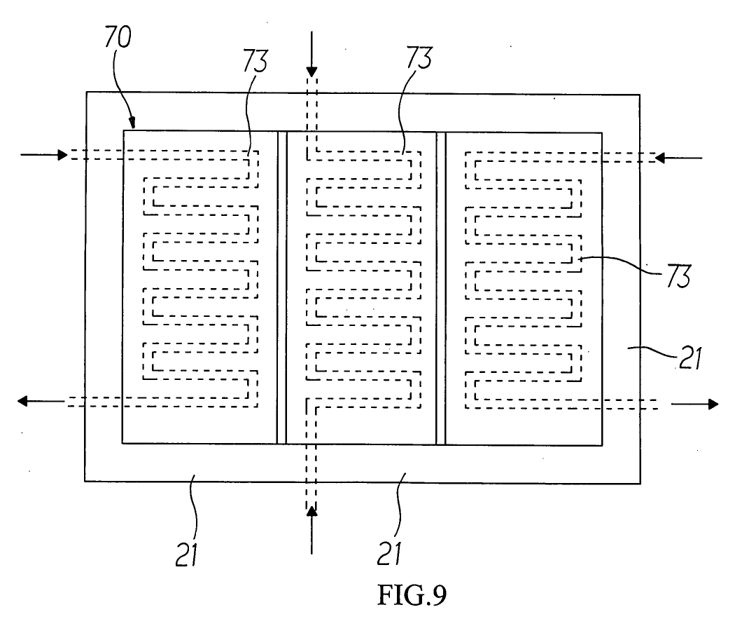

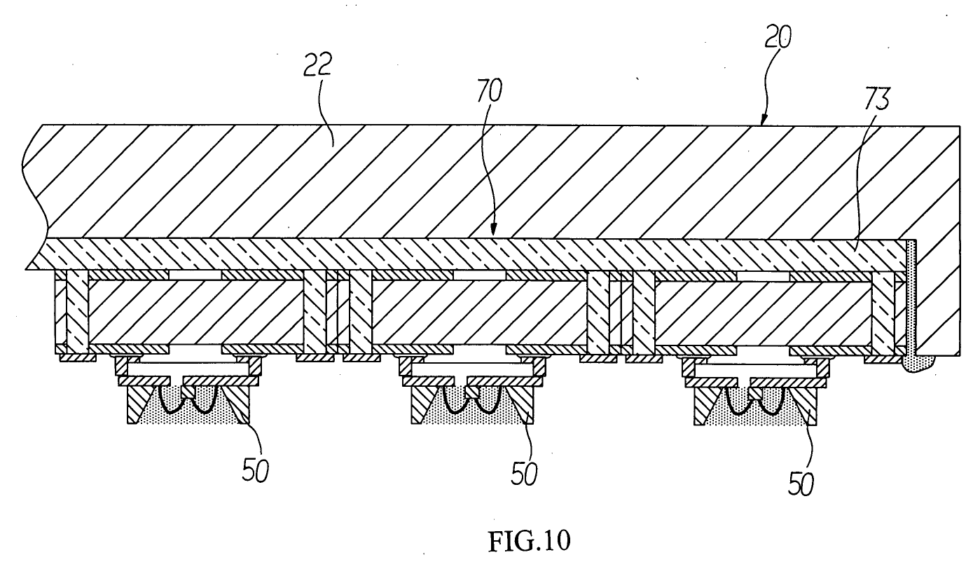

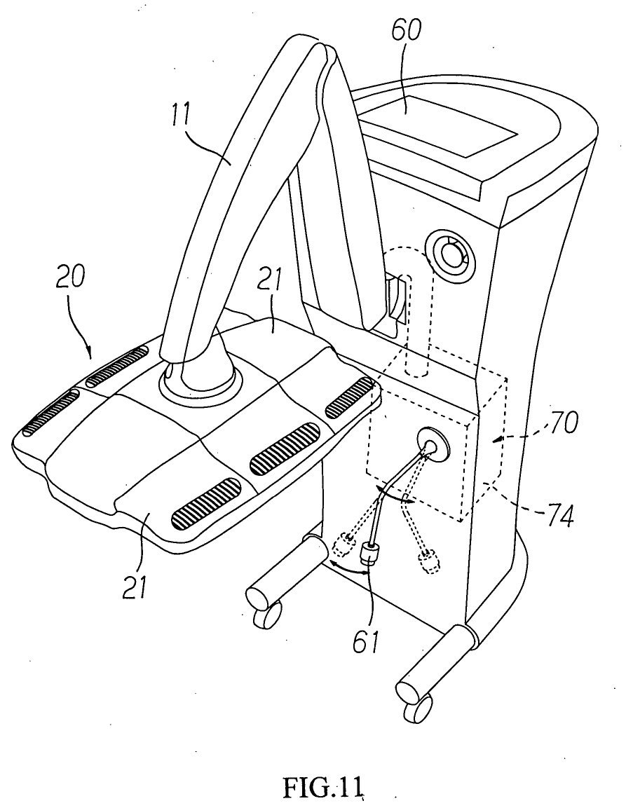

As illustrated in FIG. 7, the heat sink mechanism 70 is essentially comprised of multiple heat sink fins 71 provided on top of each irradiation panel; or alternatively, of ventilation fans 72 either on top or on both sides of each irradiation panel 21 to facilitate air convection to dissipate the heat. Of course, the heat sink mechanism 70 may be provided in the form of circulation fluid to engage in heat exchange for dissipating the heat for each irradiation panel 21 or a channel 73 is provided on each irradiation panel 21 to permit fluid circulation with all those channels 73 connecting to a pump 74 disposed at the base 10 for the fluid, e.g., cooling water or DI water to circulate among those irradiation panels 21 and the pump 74 as that done for the third preferred embodiment illustrated in FIGS. 9, 10, and 11.

A monitor system 61, e.g., CCD or CMOS, is disposed at where appropriately on the control panel 60 as illustrated in FIG. 11 to monitor the irradiation process for the target tissue while transmitting the image to any other display for observation.

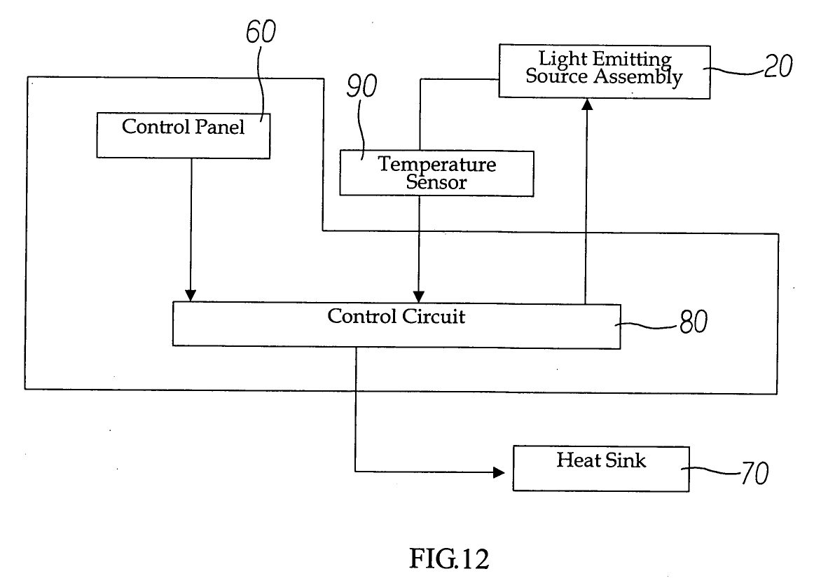

Furthermore, the control panel 60 to control the operation of the light emitting source assembly 20 in the present invention is related to a programmable panel as illustrated in FIG. 12. The control panel 60 is directly mounted to the base 10 and linked to a built-in control circuit 80 of the base 10, and further linked to those ventilation fans or the pump of the heat sink mechanism 70 as well as the light emitting source assembly 20 for precise setup of the operation of the assembly 20 through the control panel 60. For example, the control panel 60 may be programmed to control the operation time, hours, and current of each surface-mounted LED, and detect the working temperature of the assembly 20 by means of a temperature sensor 90 thus to control the rpm of the ventilation fans or the circulation rate of the fluid.

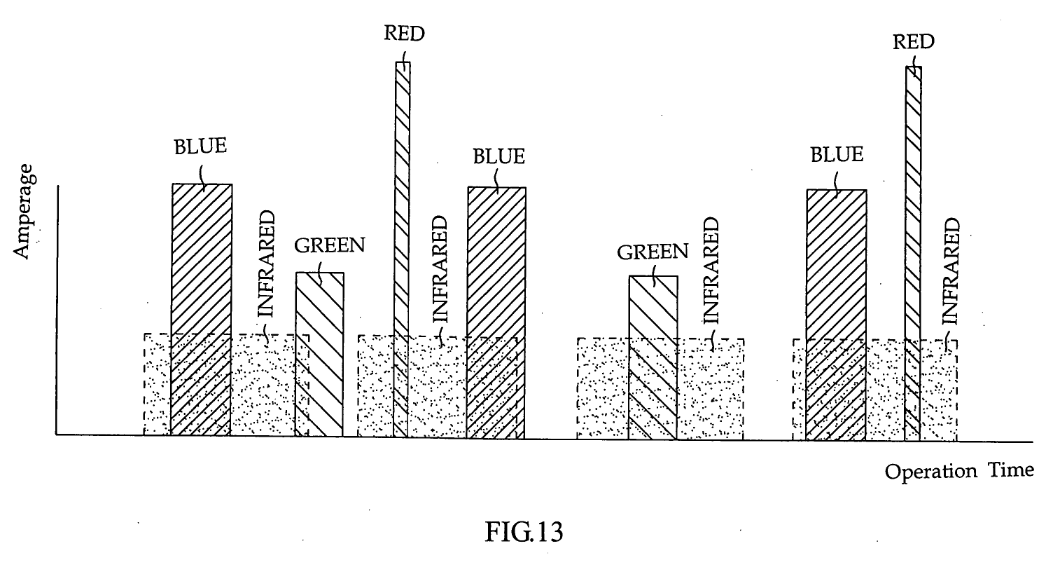

When the control panel 60 is programmed to control the operation time, hours and current of the light-emitting chip in each surface-mounted LED as illustrating in FIG. 13, a single surface-mounted LED 50 contains blue, green, red, and infrared light emitting chips and the operation time, hours, current and light emitting sequence of each light emitting chip are programmed for the control.

The prevent invention provides an improved programmable photo power source, and the application for a utility patent is duly filed accordingly. However, it is to be noted that that the preferred embodiments disclosed in the specification and the accompanying drawings are not limiting the present invention; and that any construction, installation, or characteristics that is same or similar to that of the present invention should fall within the scope of the purposes and claims of the present invention.

I claim:

1. A programmable photo power source includes a light emitting source assembly containing at least two irradiation panels foldable to each other and each comprised of multiple surface-mounted LEDs and each surface-mounted LED further containing one or multiple light emitting chips as required; and a base to secure the light emitting source assembly.

2. A programmable photo power source includes a light emitting source assembly containing at least two irradiation panels foldable to each other and each comprised of multiple surface-mounted LEDs and each surface-mounted LED further containing one or multiple light emitting chips as required; a base adapted with a support to help secure the light emitting source assembly; and a control panel to control operation time, hours and current of each surface-mounted LED of the light emitting source assembly.

3. The programmable photo power source of claim 1, wherein a reflective material is disposed on each irradiation panel of the light emitting source assembly.

4. The programmable photo power source of claim 2, wherein a reflective material is disposed on each irradiation panel of the light emitting source assembly.

5. The programmable photo power source of claim 1, wherein a heat sink mechanism is disposed on each irradiation panel of the light emitting source assembly.

6. The programmable photo power source of claim 2, wherein a heat sink mechanism is disposed on each irradiation panel of the light emitting source assembly.

7. The programmable photo power source of claim 5, wherein the heat sink mechanism is comprised of multiple heat sink fins disposed on each irradiation panel.

8. The programmable photo power source of claim 6, wherein the heat sink mechanism is comprised of multiple heat sink fins disposed on each irradiation panel.

9. The programmable photo power source of claim 5, wherein the heat sink mechanism is comprised of multiple ventilation fans disposed on each irradiation panel.

10. The programmable photo power source of claim 6, wherein the heat sink mechanism is comprised of multiple ventilation fans disposed on each irradiation panel.

11. The programmable photo power source of claim 5, wherein a channel for fluid circulation is disposed on each irradiation panel; all the channels are linked to a pump disposed at the base; and the fluid circulates among those irradiation panels and the pump.

12. The programmable photo power source of claim 6, wherein a channel for fluid circulation is disposed on each irradiation panel; all the channels are linked to a pump disposed at the base; and the fluid circulates among those irradiation panels and the pump.

13. The programmable photo power source of claim 2, wherein a control circuit is built in the base to control the circuit and link to the light emitting source assembly, and the control panel is related to a programmable panel that links to the control circuit.
